(19) United States
(12) Patent Application Publication
(10) Pub. No.: US 2026/0024667 A1
(43) Pub. Date: Jan. 22, 2026

(54) WATCHING DEVICE, WATCHING METHOD, AND PROGRAM

(71) Applicant: SEKISUI HOUSE, LTD., Osaka-shi, Osaka (JP)

(72) Inventor: Akira ICHIBOSHI, Osaka-shi, Osaka (JP)

(21) Appl. No.: 19/107,555

(22) PCT Filed: Jan. 17, 2024
(86) PCT No.: PCT/JP2024/001105

§ 371 Date: Feb. 27, 2025

(30) Foreign Application Priority Data
May 25, 2023 (JP) 2023-086378

(51) Int. Cl. G16H 50/30; A61B 5/00; G16H 10/60

(52) U.S. Cl. CPC G16H 50/30; A61B 5/7267; G16H 10/60

(57) ABSTRACT

A watching device includes a biometric information acquisition module which acquires biometric information on a person to be measured; an abnormality determination module which determines whether the person to be measured is in an abnormal state based on the biometric information; a reporting module which reports, when the person to be measured is determined to be in the abnormal state, that the person to be measured is in the abnormal state; a state determination module which determines whether the person to be measured is in a predetermined temporary physical state; and a reporting restriction module which restricts the reporting by the reporting module when the person to be measured is determined to be in the temporary physical state.

### TECHNICAL FIELD

The present invention relates to a watching device, a watching method, and a program, and more particularly to, a technology of determining a physical abnormality of a person to be measured based on biometric information on the person to be measured.

### BACKGROUND ART

There has been known a system which detects biometric information on a person to be measured, such as a heart rate and a respiratory rate, during sleep or the like. For example, in Patent Literature 1, there is disclosed an abnormality evaluation device which acquires the respiratory rate of the person to be measured during sleep, and compares the acquired respiratory rate with a reference respiratory rate, to thereby determine whether or not the person to be measured is in a physically abnormal state. In this device, when the person to be measured is determined to be in the abnormal state, this fact is reported to the person to be measured and a nurse.

### CITATION LIST

### Patent Literature

[Patent Literature 1] JP 6193650 B2

### SUMMARY OF INVENTION

### Technical Problem

The heart rate and the respiratory rate of the person to be measured greatly change after drinking or after exercising although such changes are temporary. Thus, with the above-mentioned related-art technology, there is a possibility that the person to be measured is determined to be in the abnormal state in the measurement after drinking or after exercising and thus the fact of the abnormal state is reported.

The present invention has been made in view of the above-mentioned problem, and has an object to provide a watching device, a watching method, and a program which are capable of appropriately reporting an abnormality of a person to be measured in consideration of a temporary physical state of the person to be measured such as drinking and exercising.

### Solution to Problem

(1) In order to solve the above-mentioned problem, a watching device according to one embodiment of the present invention includes: biometric information acquisition means for acquiring biometric information on a person to be measured; abnormality determination means for determining whether the person to be measured is in an abnormal state based on the biometric information; reporting means for reporting, when the person to be measured is determined to be in the abnormal state, that the person to be measured is in the abnormal state; state determination means for determining whether the person to be measured is in a predetermined temporary physical state; and reporting restriction means for restricting the reporting by the reporting means when the person to be measured is determined to be in the predetermined temporary physical state.
(2) In the watching device according to the above-mentioned item (1), the reporting restriction means may be configured to change a determination criterion to be used in the abnormality determination means when the person to be measured is determined to be in the predetermined temporary physical state.
(3) In the watching device according to the above-mentioned item (2), the watching device may further include influence level determination means for determining an influence level of the predetermined temporary physical state for the person to be measured. The reporting restriction means may be configured to change the determination criterion to be used in the abnormality determination means in accordance with the influence level.
(4) In the watching device according to any one of the above-mentioned items (1) to (3), when the person to be measured is determined to be in the predetermined temporary physical state, the reporting restriction means may be configured to inhibit execution of the reporting by the reporting means until the person to be measured is determined not to be in the predetermined temporary physical state.
(5) In the watching device according to any one of the above-mentioned items (1) to (4), the state determination means may be configured to determine whether the person to be measured is in the predetermined temporary physical state based on the biometric information.
(6) In the watching device according to the above-mentioned item (5), the state determination means may include a machine learning model trained through use of the biometric information on a person who is in the predetermined temporary physical state.
(7) In the watching device according to any one of the above-mentioned items (1) to (6), the predetermined temporary physical state may be a state after drinking or a state after exercising.
(8) Further, a watching method according to one embodiment of the present invention includes: a biometric information acquisition step of acquiring biometric information on a person to be measured; an abnormality determination step of determining whether the person to be measured is in an abnormal state based on the biometric information; a reporting step of reporting, when the person to be measured is determined to be in the abnormal state, that the person to be measured is in the abnormal state; a state determination step of determining whether the person to be measured is in a predetermined temporary physical state; and a reporting restriction step of restricting the reporting in the reporting step when the person to be measured is determined to be in the predetermined temporary physical state.
(9) A program according to one embodiment of the present invention is a program for causing a computer to function as: biometric information acquisition means for calculating biometric information on a person to be measured; abnormality determination means for determining whether the person to be measured is in an abnormal state based on the biometric information; reporting means for reporting, when the person to be measured is determined to be in the abnormal state, that the person to be measured is in the abnormal state; state determination means for determining whether the person to be measured is in a predetermined temporary physical state; and reporting restriction means for restricting the reporting by the reporting means when the person to be measured is determined to be in the predetermined temporary physical state. This program may be stored in a computer-readable information storage medium such as a semiconductor memory or a magneto-optical disk.

### Advantageous Effects of Invention

According to the present invention, it is possible to appropriately report the abnormality of the person to be measured in consideration of the temporary physical state of the person to be measured such as drinking and exercising.

### BRIEF DESCRIPTION OF DRAWINGS

FIG. 1 is an overall configuration diagram of a watching system in an embodiment of the present invention.

FIG. 2 is a functional block diagram of a watching device according to the embodiment of the present invention.

FIG. 3 is a flowchart for illustrating an operation example of the watching device.

FIG. 4 is a flowchart for illustrating a modified operation example of the watching device.

### DESCRIPTION OF EMBODIMENTS

Now, an embodiment of the present invention is described in detail with reference to the accompanying drawings.

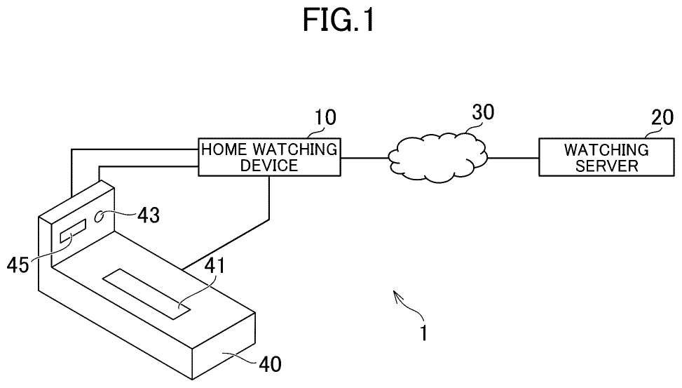

FIG. 1 is an overall configuration diagram of a watching system in the embodiment of the present invention. A watching system 1 of FIG. 1 is configured such that the watching system 1 is centered around a bed 40 installed in a house. To a headboard of the bed 40, a speaker/microphone 43 and a Doppler sensor 45 are mounted. The Doppler sensor 45 emits a microwave toward the chest of a person to be measured sleeping in the bed 40, and receives a reflected wave thereof. From the reflected wave, a Doppler signal indicating a motion of the chest caused by heartbeat and respiration is generated, and Doppler data obtained by digitizing this Doppler signal is output. The speaker/microphone 43 is also provided such that the speaker/microphone 43 is directed toward the person to be measured sleeping in the bed 40. Moreover, a weight scale 41 is mounted to a floorboard of the bed 40 or the like, and measures the weight of the person to be measured, bedding, and the like. The weight scale 41, the speaker/microphone 43, and the Doppler sensor 45 are connected to a watching device 10 installed in the same house.

The watching device 10 acquires biometric information (here, a heart rate and a respiratory rate) of the person to be measured (not shown) sleeping in the bed 40 based on the Doppler data measured by the Doppler sensor 45, and detects an abnormality of the person to be measured based on this biometric information. When the abnormality is detected, a call message such as “Abnormality is detected. Are you all right?” is acoustically output from a speaker portion of the speaker/microphone 43. When a response such as “I′m all right.” is not input from the person to be measured to a microphone portion of the speaker/microphone 43 in response to this call, the watching device 10 transmits the fact of the abnormality to a watching server 20 connected via a communication network 30 such as the Internet. The watching server 20 is a computer installed in a watching center remotely provided. When the watching server 20 receives the fact of the abnormality, a staff member of the watching center again executes the call and the like from the speaker portion of the speaker/microphone 43, and requests dispatch of a medical worker such as a medical doctor or an ambulance car to this house as required.

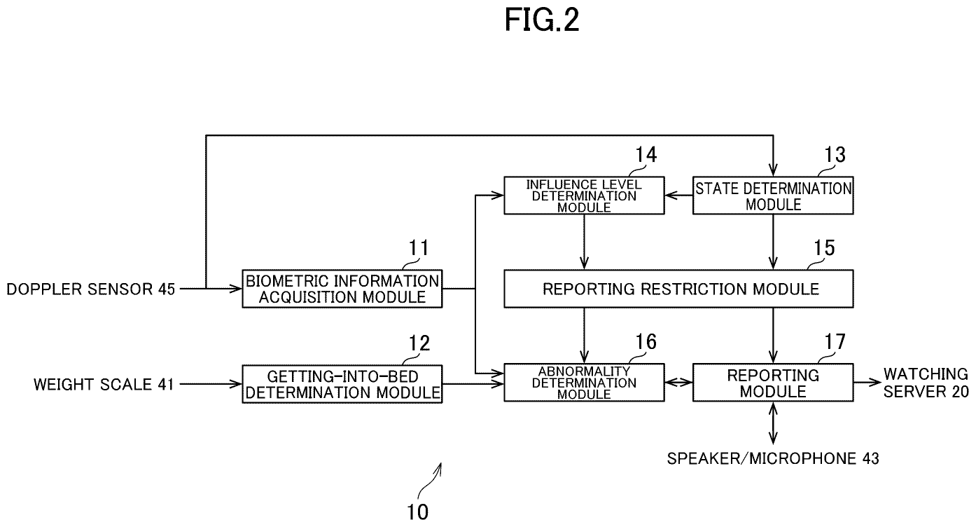

FIG. 2 is a functional block diagram of the watching device 10 according to the embodiment of the present invention. As illustrated in FIG. 2, the watching device 10 functionally includes a biometric information acquisition module 11, a getting-into-bed determination module 12, a state determination module 13, an influence level determination module 14, a reporting restriction module 15, an abnormality determination module 16, and a reporting module 17. The watching device 10 includes a general-purpose computer including a CPU and a memory. Each function of FIG. 2 is implemented by executing a program according to the embodiment of the present invention in this computer. This program may be supplied from a computer-readable storage medium such as a semiconductor memory to the computer, or may be supplied to the computer by downloading this program from another computer via the communication network 30 such as the Internet.

The biometric information acquisition module 11 acquires the biometric information on the person to be measured based on the Doppler data detected by the Doppler sensor 45. In this case, the heart rate and the respiratory rate are acquired as the biometric information. For example, peaks corresponding to the heart rate and the respiratory rate are identified by applying Fourier analysis to the Doppler data, and the heart rate and the respiratory rate are acquired from positions (frequencies) of those peaks.

The getting-into-bed determination module 12 determines whether or not the person to be measured is sleeping in the bed 40 based on the weight detected by the weight scale 41. For example, a body weight of the person to be measured is stored in advance, and the person to be measured is determined to have got into bed at a timing at which the weight detected by the weight scale 41 increases by the body weight. Moreover, the person to be measured is determined to have left his or her bed at a timing at which the weight detected by the weight scale 41 decreases by the body weight stored in advance.

The state determination module 13 determines whether or not the person to be measured is in a predetermined temporary physical state. Herein, “predetermined temporary physical state” is a state after drinking and a state after exercising. It is determined whether the person to be measured is in the state after drinking (state in which influence of drinking remains), in the state after exercising (state in which influence of exercise remains), in a usual state, or in another state based on the Doppler data detected by the Doppler sensor 45. As an example, this determination may be made through use of a machine learning model. Specifically, learning data is created by adding a label indicating the state after drinking to the Doppler data of a certain period (as an example, five minutes) of a person who is in the state after drinking or a feature amount thereof. Moreover, learning data is created by adding a label indicating the state after exercising to the Doppler data of a certain period of a person who is in the state after exercising or a feature amount thereof. Further, learning data is created by adding a label indicating the usual state to the Doppler data of a certain period of a person who is in the usual state (which corresponds to none of the state after drinking and the state after exercising) or a feature amount thereof. After that, the machine learning model which classifies the physical state from the Doppler data or the feature amount thereof is trained through use of those pieces of learning data. There is a tendency that in the state after drinking, the heart rate and the respiratory rate are high and intensities thereof are also high. Moreover, changes in heart rate and respiratory rate tend to be small after falling asleep. Moreover, regarding the respiration, a ratio between exhalation and inhalation tends to change. Meanwhile, there is a tendency that in the state after exercising, the heart rate and the respiratory rate are high and the intensities thereof are also high. Moreover, the heart rate and the respiratory rate tend to gradually decrease after falling asleep. Regarding the respiration, the ratio between the exhalation and the inhalation tends to fall within a certain range. It is possible to determine whether the person to be measured is in the state after drinking, in the state after exercising, in the usual state, or in the other state by causing the machine learning model to learn such features.

In the above, the temporary physical state of the person to be measured is determined by the machine learning model, but may be determined by another method. For example, a sensor may be provided to a dining table or a refrigerator, and it may be determined whether or not the person to be measured is in the state after drinking from a detection result obtained by the sensor. Moreover, an exhalation sensor may be provided, and it may be determined whether or not the person to be measured is in the state after drinking from an alcohol concentration contained in the exhalation. Moreover, a video camera may be provided close to the dining table in the house, and it may be determined, from content captured by the video camera, whether or not the person to be measured has drunk alcohol. Moreover, a video camera may be provided in a living room, and it may be determined, from content captured by the video camera, whether or not the person to be measured has done exercise in the house. Moreover, the person to be measured himself or herself may be prompted to input, to the watching device 10, whether the person to be measured has drunk alcohol, or has done exercise.

When the person to be measured is in the state after drinking, the influence level determination module 14 determines an influence level thereof (magnitude of influence of drinking). Moreover, when the person to be measured is in the state after exercising, the influence level determination module 14 determines an influence level thereof (magnitude of influence of exercise). For example, as the influence of drinking that remains after drinking becomes greater, the heart rate and the respiratory rate of the person to be measured become higher. Thus, a range of the heart rate and/or a range of the respiratory rate is set for each influence level (for example, for each of three levels). The influence level determination module 14 checks the range of the influence level to which the heart rate and/or the respiratory rate of the person to be measured acquired by the biometric information acquisition module 11 belongs when the state determination module 13 determines that the person to be measured is in the state after drinking, to thereby determine the influence level of the drinking.

Further, as the influence of exercise that remains after exercising becomes greater, the heart rate and the respiratory rate of the person to be measured become higher. Thus, a range of the heart rate and/or a range of the respiratory rate is set in advance for each influence level (for example, for each of three levels). The influence level determination module 14 may check the range of the influence level to which the heart rate and/or the respiratory rate of the person to be measured acquired by the biometric information acquisition module 11 belongs when the state determination module 13 determines that the person to be measured is in the state after exercising, to thereby determine the influence level of the exercise.

The abnormality determination module 16 determines whether or not the person to be measured is in the abnormal state based on the heart rate and the respiratory rate of the person to be measured acquired by the biometric information acquisition module 11. As an example, a plurality of numerical ranges of the heart rate are provided, and a risk value is set to each numerical range. Moreover, a risk value relating to the heart rate is determined by checking the numerical range to which the heart rate belongs. Similarly, a plurality of numerical ranges of the respiratory rate are provided, and a risk value is set to each numerical range. Moreover, a risk value relating to the respiratory rate is determined by checking the numerical range to which the respiratory rate belongs. After that, the abnormality determination module 16 calculates a total risk value by adding the risk value relating to the heart rate and the risk value relating to the respiratory rate to each other. When this total risk value is equal to or higher than a given threshold value, it is determined that the person to be measured is in the abnormal state. As the given threshold value, there are prepared a usual threshold value used when the person to be measured is in the usual state and a post-drinking threshold value used when the person to be measured is in the state after drinking. As described later, a plurality of types of post-drinking threshold values may be prepared in accordance with the degree (influence level) of the remaining influence of drinking. Moreover, it may be determined whether or not the person to be measured is in the abnormal state also when the person to be measured is in the state after exercising. In this case, a post-exercise threshold value may further be prepared. A plurality of types of post-exercise threshold values may also be prepared in accordance with the degree (influence level) of the remaining influence of exercise.

When the person to be measured is in the abnormal state, the reporting module 17 reports this fact. Specifically, the reporting module 17 acoustically outputs a call message from the speaker portion of the speaker/microphone 43. Moreover, when a response from the person to be measured is not input to the microphone portion of the speaker/microphone 43 in response thereto, the reporting module 17 transmits a message indicating that the person to be measured is in the abnormal state to the watching server 20 via the communication network 30.

The reporting restriction module 15 restricts the reporting by the reporting module 17 when the person to be measured is determined to be in the state after drinking or the state after exercising. For example, when the person to be measured is in the state after drinking, the reporting restriction module 15 changes a determination criterion for the abnormal state to be used in the abnormality determination module 16. Specifically, the reporting restriction module 15 changes the above-mentioned threshold value to be compared with the total risk value to the post-drinking threshold value increased by a predetermined value from the usual threshold value. As a result, it is less likely that the total risk value becomes equal to or higher than the threshold value, and hence the determination of the abnormal state is made. In this manner, the reporting by the reporting module 17 can be suppressed. In this case, the degree of increase in threshold value may be changed in accordance with the influence level of drinking. Specifically, as the influence level of drinking becomes higher, a larger post-drinking threshold value may be used. With this configuration, the determination criterion can appropriately be set in accordance with the influence level of drinking. The reporting restriction module 15 may stop the abnormality determination by the abnormality determination module 16 or may stop the reporting by the reporting module 17 until the person to be measured is determined not to be in the state after drinking.

Similarly, also when the person to be measured is in the state after exercising, the reporting restriction module 15 may change a determination criterion for the abnormal state to be used in the abnormality determination module 16. Specifically, the reporting restriction module 15 changes the above-mentioned threshold value to be compared with the total risk value to the post-exercise threshold value increased by a predetermined value from the usual threshold value. In this case, the degree of increase in threshold value may be changed in accordance with the influence level of exercise. Specifically, as the influence level of exercise becomes higher, a larger post-exercise threshold value may be used. Alternatively, the reporting restriction module 15 may stop the abnormality determination by the abnormality determination module 16 or may stop the reporting by the reporting module 17 until the person to be measured is determined not to be in the state after exercising.

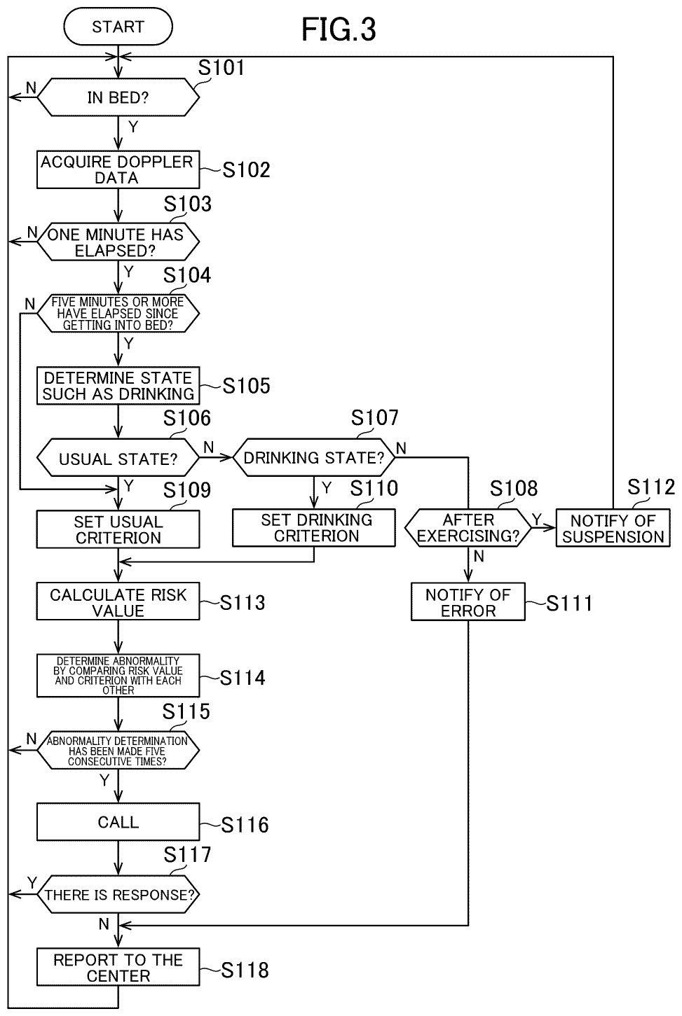

FIG. 3 is a flowchart illustrating an operation example of the watching device 10. As illustrated in FIG. 3, in the watching device 10, the getting-into-bed determination module 12 first monitors whether or not the person to be measured has got into the bed 40 (Step S101). When the person to be measured has got into bed 40, the biometric information acquisition module 11 then acquires the Doppler data transmitted from the Doppler sensor 45 (Step S102). The watching device 10 repeats the processing steps of Step S101 and Step S102 until one minute elapses (Step S103). When one minute has elapsed (Step S103), the watching device 10 then determines whether or not five minutes or longer have elapsed since the person to be measured got into the bed (Step S104). When five minutes or longer have not elapsed, the abnormality determination module 16 sets the usual threshold value as the determination criterion (Step S109).

When the watching device 10 determines that five minutes or longer have elapsed in Step S104, the state determination module 13 determines the state of the person to be measured and the influence level determination module 14 determines the influence levels of the drinking and the like (Step S105). Specifically, the state determination module 13 determines whether or not the person to be measured is in the state after drinking or exercising by inputting the Doppler data of the last five minutes to the machine learning model. As a result, when the state determination module 13 determines that the person to be measured is in the usual state (Step S106), the abnormality determination module 16 sets the usual threshold value as the determination criterion (Step S109). Moreover, when the state determination module 13 determines that the person to be measured is in the state after drinking (Step S107), the abnormality determination module 16 sets, as the determination criterion, the post-drinking threshold value corresponding to the influence level of drinking (Step S110). Moreover, when the state determination module 13 determines that the person to be measured is in the state after exercising (Step S108), the reporting module 17 acoustically outputs, from the speaker portion of the speaker/microphone 43, the fact that the abnormality determination is to be temporarily stopped (suspended) (Step S112), and the process returns to Step S101. Moreover, when the state determination module 13 determines that the person to be measured is not in the state after exercising (Step S108), the reporting module 17 acoustically outputs, from the speaker portion of the speaker/microphone 43, the fact that the abnormality determination is to be stopped (Step S111), and transmits a message indicating this fact to the watching server 20.

After that, the abnormality determination module 16 calculates the total risk value based on the heart rate and the respiratory rate acquired by the biometric information acquisition module 11 (Step S113). Then, the abnormality determination module 16 compares the total risk value with the threshold value set in Step S109 or Step S110, to thereby make the abnormality determination (Step S114).

When the abnormality determination module 16 determines that the person to be measured is in the abnormal state in the last five consecutive abnormality determinations (Step S115), the reporting module 17 executes the call through use of the speaker portion of the speaker/microphone 43 (Step S116). When the response of the person to be measured to this call is acoustically collected by the microphone portion of the speaker/microphone 43 (Step S117), the process returns to Step S101. Moreover, when the response of the person to be measured is not collected (Step S117), the reporting module 17 transmits, to the watching server 20, a message indicating that the abnormality has occurred in the person to be measured (Step S118), and the process returns to Step S101.

Moreover, in Step S115, also when the abnormality determination module 16 determines that the person to be measured is not in the abnormal state in the last five consecutive abnormality determinations, the process returns to Step S101. Then, the state determination module 13 again determines, based on the Doppler data of the last five minutes, the state of the person to be measured after one minute has elapsed, and the influence level determination module 14 determines the influence levels of drinking and the like (Step S105). The watching device 10 sets the usual threshold value as the determination criterion (Step S109) when the person to be measured is determined to be in the usual state (Step S106), which has transitioned from the state after drinking (Step S107), and continues the subsequent processing steps.

With the watching system 1 described above, the heart rate and the respiratory rate of the person to be measured can be acquired after getting into bed or during sleeping based on the Doppler data acquired by the Doppler sensor 45, and the total risk value is calculated from the acquired values. The total risk value is compared with the given threshold value. When the total risk value is equal to or higher than the threshold value, it is determined that the abnormality has occurred. The reporting module 17 reports this fact to the person to be measured himself or herself or the staff member of the watching center. In this embodiment, it is determined, from the Doppler data, whether or not the person to be measured is in the temporary physical state caused by drinking or exercising, to thereby restrict the reporting by the reporting module 17. Thus, it is possible to inhibit excessive reporting to the person to be measured himself or herself or the staff member of the watching center.

The present invention is not limited to the embodiment described above, and various modifications may be made thereto. Such modifications also belong to the technical scope of the present invention.

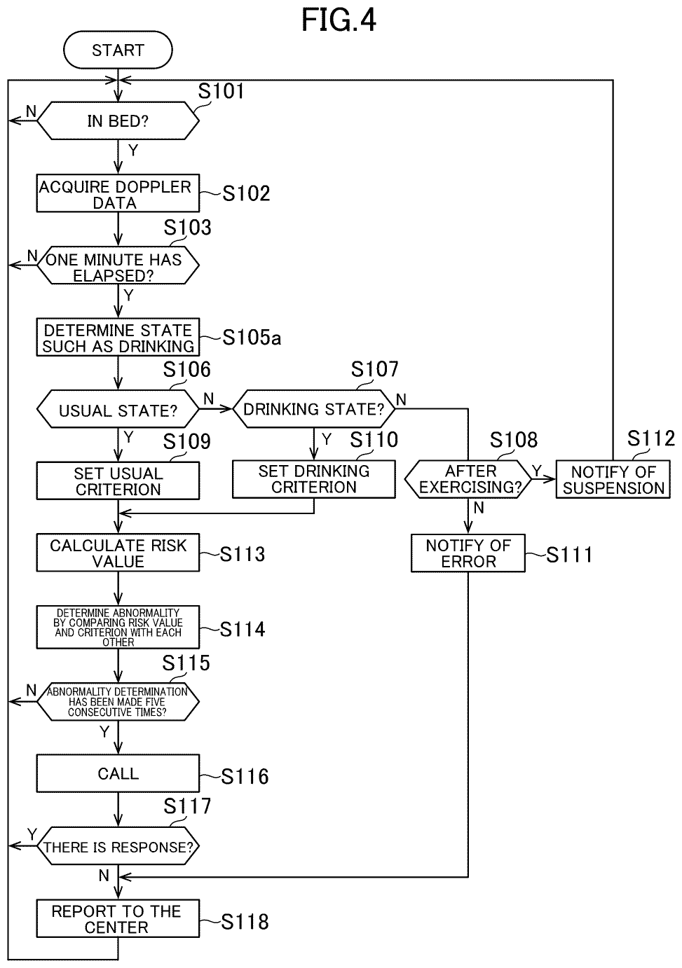

For example, in the operation example of FIG. 3, the determination of the state such as drinking is not made until five minutes or longer have elapsed since the person to be measured got into bed, but the determination of the state such as drinking may be made when one minute has elapsed since the person to be measured got into bed. FIG. 4 is a flowchart for illustrating an operation of the watching device in this case. As illustrated in FIG. 4, when one minute has elapsed since the person to be measured got into bed, the state determination module 13 determines the state of the person to be measured, and the influence level determination module 14 determines the influence levels of drinking and the like (Step S105a). At this time, when a time that has elapsed since the person to be measured got into bed is shorter than five minutes, the state determination module 13 determines the state of the person to be measured through use of, for example, a first machine learning model based on the Doppler data of the last one minute. Moreover, when five minutes or longer have elapsed since the person to be measured got into bed, the state determination module 13 determines the state of the person to be measured through use of, for example, a second machine learning model based on the Doppler data of the last five minutes. When the first machine learning model is to be created, the learning data is created by adding a label indicating the state such as the state after drinking to the Doppler data of one minute on a person who is in each of the state after drinking, the state after exercising, and the usual state or the feature amount thereof. It is only required to use the learning data created as described above to train the first machine learning model which classifies the physical state from the Doppler data of one minute or the feature amount thereof. Similarly, when the second machine learning model is to be created, the learning data is created by adding a label indicating the state such as the state after drinking to the Doppler data of five minutes on a person who is in each of the state after drinking, the state after exercising, and the usual state or the feature amount thereof. It is only required to use the learning data created as described above to train the second machine learning model which classifies the physical state from the Doppler data of five minutes or the feature amount thereof. With this modification example, it is possible to appropriately make the abnormality determination in consideration of the temporary state of the person to be measured when one minute has elapsed since the person to be measured got into bed.

### REFERENCE SIGNS LIST

1 watching system, 10 watching device, 11 biometric information acquisition module, 12 getting-into-bed determination module, 13 state determination module, 14 influence level determination module, 15 reporting restriction module, 16 abnormality determination module, 17 reporting module, 20 watching server, 30 communication network, 40 bed, 41 weight scale, 43 speaker/microphone, 45 Doppler sensor

The invention claimed is:

1. A watching device, comprising:
at least one processor; and
at least one memory device storing instructions which, when executed by the at least one processor, causes the at least one processor to perform operations including:
acquiring biometric information on a person to be measured;
determining whether the person to be measured is in an abnormal state based on the biometric information;
reporting, when the person to be measured is determined to be in the abnormal state, that the person to be measured is in the abnormal state;
determining whether the person to be measured is in a predetermined temporary physical state; and
restricting the reporting when the person to be measured is determined to be in the predetermined temporary physical state.

2. The watching device according to claim 1, wherein restricting comprises changing a determination criterion to be used in determining whether the person to be measured is in the abnormal state when the person to be measured is determined to be in the predetermined temporary physical state.

3. The watching device according to claim 2, the operations further comprising:
determining an influence level of the predetermined temporary physical state for the person to be measured, and
wherein reporting comprises changing the determination criterion to be used in determining whether the person to be measured is in the abnormal state, in accordance with the influence level.

4. The watching device according to claim 1, wherein, when the person to be measured is determined to be in the predetermined temporary physical state, reporting comprises inhibiting execution of the reporting until the person to be measured is determined not to be in the predetermined temporary physical state.

5. The watching device according to claim 1, wherein determining whether the person to be measured is in the predetermined temporary physical state based on the biometric information.

6. The watching device according to claim 5, wherein determining whether the person to be measured is in the predetermined temporary physical state by using a machine learning model trained through use of the biometric information on a person who is in the predetermined temporary physical state.

7. The watching device according to claim 6, wherein the predetermined temporary physical state is a state after drinking or a state after exercising.

8. A watching method, comprising:
acquiring biometric information on a person to be measured;
determining whether the person to be measured is in an abnormal state based on the biometric information;
reporting, when the person to be measured is determined to be in the abnormal state, that the person to be measured is in the abnormal state;
determining whether the person to be measured is in a predetermined temporary physical state; and
restricting the reporting when the person to be measured is determined to be in the predetermined temporary physical state.

9. An information storage medium storing a program for causing a computer to execute:
calculating biometric information on a person to be measured;
determining whether the person to be measured is in an abnormal state based on the biometric information;
reporting, when the person to be measured is determined to be in the abnormal state, that the person to be measured is in the abnormal state;
determining whether the person to be measured is in a predetermined temporary physical state; and
restricting the reporting when the person to be measured is determined to be in the predetermined temporary physical state.
